(19) United States
(12) Patent Application Publication
(10) Pub. No.: US 2004/0199155 A1
(43) Pub. Date: Oct. 7, 2004

(54) Devices and methods for repair of valves in the human body

(75) Inventor: Kenneth H. Mollenauer, Saratoga, CA (US)

Correspondence Address: CROCKETT & CROCKETT, 24012 CALLE DE LA PLATA, SUITE 400, LAGUNA HILLS, CA, 92653, US

(73) Assignee: Starion Instruments, Inc.

(21) Appl. No.: 10/830,920

(22) Filed: Apr. 23, 2004

Related U.S. Application Data
(63) Continuation of application No. 09/598,852, filed on Jun. 20, 2000, now abandoned.

(51) Int. Cl. A61B 18/04; A61F 7/12

(52) U.S. Cl. 606/27; 604/113

(57) ABSTRACT

Devices and method for treating various incompetent anatomical valves by thermally damaging the nearby supporting tissue of the body vessel controlled by the valve.

This application is a continuation of U.S. application Ser. No. 09/598,852, filed Jun. 20, 2000.

### FIELD OF THE INVENTIONS

The inventions described below relate to the fields of minimally invasive surgery, vascular surgery, urology and general surgery.

### BACKGROUND OF THE INVENTIONS

Several diseases of the human body result from poor performance of natural valves within the body. Venous insufficiency, gastrointestinal reflux, and urinary incontinence are examples of maladies which are the result of the failure of valves to perform normally.

Venous insufficiency generally refers to conditions which cause the veins in the leg to become incapable of functioning properly due to failure of venous valves. Generally, the return of blood from the veins in the leg to the heart is caused by the interaction of the leg muscles with venous valves that function as check valves. Muscular activity in the legs forces venous blood upward toward the heart, through the venous valves which are generally found in the perforating veins between the superficial veins and the deep veins in the leg.

Venous insufficiency is often caused by failure of the valves located in small communicating veins which connect large superficial veins such as the posterior arch vein or saphenous vein, with large deep veins such as the peroneal or tibial veins. The communicating veins have valves which look like duckbill valves or leaflet valves that act as check valves, allowing blood to flow from the superficial veins into the deep veins, but blocking flow in the reverse direction. Exercise and movement of the calf muscles around the communicating veins squeezes blood through the communicating veins. This mechanical pumping action, combined with the function of the valves, is responsible for returning blood flow to the heart.

When the valves fail, venous blood in the superficial veins cannot be pumped into the deeper veins, resulting in blood pooling in the legs. The condition causes very poor circulation in the legs and can lead to varicose veins and skin ulcers. Large varicose veins in the lower leg, skin ulcers just above the ankle bone on the inside of the calf, and discolored skin on the lower leg, are common symptoms. Similar symptoms are seen in other areas of the body, particularly the thighs and arms, when perforating veins in those areas become incompetent.

Venous insufficiency is generally attributed to the failure of certain groups of perforating veins. There are about 150 perforating veins in the leg, but there are several major perforating veins which are important contributors to the problem of venous insufficiency. An important group of perforators is found high on the inside of the calf, over the calf muscle. Another important group of perforators is found low on the inside of the calf, just above the ankle and toward the back of the leg. Another set of perforators is found on the lateral or outside of the leg run though the muscles on the outside of the calf.

An effective surgical treatment of this condition was developed by Linton circa 1938. In the Linton procedure, also referred to as the Medial subfascial approach, the calf is cut open along the Linton line, extending from just above the ankle bone (or medial malleolus) on the inside or medial side of the foot, up the inside of the calf almost to the knee. The incision is deep enough to cut the skin and fat, and also the deep fascia which is a filmy fibrous layer of tissue which covers the muscles of the calf. Upon peeling away the skin, fat and fascia, some of the communicating veins can be seen, and these are cut and tied off. A minimally invasive version of this procedure is described in U.S. Pat. No. 5,979,452.

An alternative to the Linton procedure is repair of the veins. One method for repairing the venous valves is disclosed in Farley, et al., Catheter Having Expandable Electrodes And Adjustable Stent, U.S. Pat. No. 6,014,589 (Jan. 11, 2000). Farley proposes a catheter having expandable electrodes for applying energy to a vein and having expandable stent members for limiting vein shrinkage to a final desired vein diameter. The catheter includes a set of expandable arms that are pre-formed into an outwardly bowed configuration. An electrode is mounted on each arm. The catheter is delivered percutaneously into the veins of a patient, and positioned near a failed valve. The stent arms are expanded outward to the desired final diameter of the vein. The electrode arms are then expanded into apposition with the vein wall and energy is applied to shrink the vein into contact with the stent arms.

Urinary incontinence is another condition caused, at least in part, by failure of an anatomical valve. Stress Urinary incontinence, or SUI, effects females and results from the inoperability of the bladder neck sphincter, which controls flow or urine from the bladder. Various treatments have been tried, and the bladder neck suspension is the predominant surgical cure. Bladder neck suspension methods include various procedures for lifting the bladder neck and urging it anteriorly, toward the front of the body. The bladder neck is tied to another structure in the body, and is literally suspended from these structures. This suspension alters the forces countering the closure of the bladder neck sphincter, and cures the condition. Bladder neck suspension may be accomplished with open surgical techniques or minimally invasive techniques. Minimally invasive techniques, such as those indicated in Benderev, U.S. Pat. No. 5,860,425, require penetration of the skin and insertion of various suturing and knot tying devices into the body.

Gastroesophageal reflux disease, or GERD, is another condition caused, at least in part, by failure of an anatomical valve. In GERD, the stomach contents are regurgitated from the stomach into the lower esophagus due to a failure of the lower esophageal sphincter. Since the stomach contents are highly acidic, the condition is uncomfortable and, if left untreated, potentially dangerous. Symptoms range from mere heartburn to pulmonary disorders, ulcer formation, or esophagitis esophageal obstruction and perforation of the esophagus. One surgical treatment of GERD is the Nissen fundoplication, in which a surgeon constructs a new valve to support LES by pulling the gastric fundis upward and wrapping it around the lower esophagus. The procedure is accomplished through open surgery, but a version of the Nissen fundoplication can now be accomplished with minimally invasive techniques. Recently, RF ablation of the lower esophageal sphincter has been proposed, under the assumption that aberrant electrical activity in the lower esophageal sphincter causes the reflux, and the ablation of aberrant electrical tissue will reduce lower esophageal sphincter relaxations.

### SUMMARY

The devices and methods described below provide for the treatment of various incompetent valves and sphincters throughout the body. The catheters provide for location of a heating element or other tissue necrosing tool in the lumen of the vessel controlled by the valve, at or near the base of the valve (but not on the valve itself). Additionally, the catheters include balloons for locating and anchoring the distal section of the catheter within the lumen, such that the heating element is positioned near the base of the valve, in contact with lumenal tissue at the base of the valve. The catheters also include suction ports on the distal end of the catheter which can be operated to size or draw down the vessel to the diameter of the catheter, so that the vessel walls are in contact with the heating elements. The catheters may be used for treatment of venous insufficiency, urinary incontinence, and gastroesophageal reflux disease.

### BRIEF DESCRIPTION OF THE DRAWINGS

FIG. 1 illustrates a venous repair catheter positioned with a vein, in the proximity of a venous valve.

FIG. 2 illustrates a venous repair catheter in one step of operation, drawing the vein segment to be treated toward the catheter body.

FIG. 3 illustrates the segment of diseased vein after treatment and withdrawal of the venous repair catheter.

FIG. 4 illustrates a venous repair catheter positioned within a vein, where the catheter is adapted to treat a vein segment including several valves in a single application.

FIG. 5 illustrates the anatomy involved in stress urinary incontinence in a female patient.

FIG. 6 illustrates the repair catheter adapted for use in treatment of stress urinary incontinence in a female patient.

FIG. 7 illustrates the anatomy involved in gastroesophageal reflux disease.

FIG. 8 illustrates the repair catheter of FIG. 7 in use in the treatment or gastroesophageal reflux disease in a patient.

### DETAILED DESCRIPTION OF THE INVENTIONS

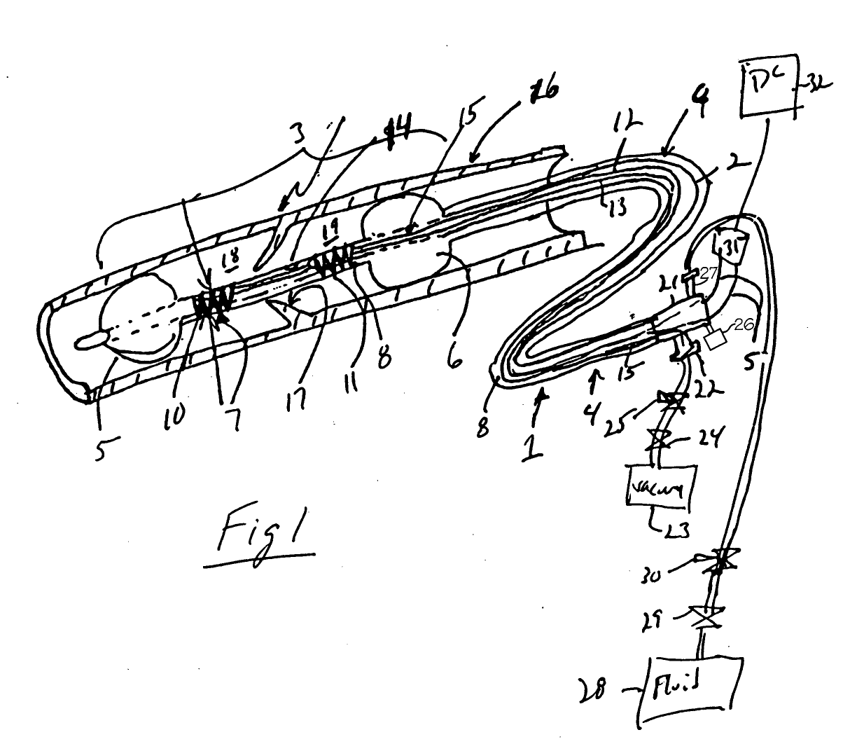

FIG. 1 illustrates a venous repair catheter positioned within a vein, in the proximity of a venous valve that is to be repaired. The catheter 1 includes a tubular catheter body 2 characterized by a distal section 3 and a proximal section 4. The distal section is adapted for percutaneous insertion into the blood vessel, and the proximal end, which remains outside the body, is adapted for connection to various external systems, such as to vacuum source, inflation reservoirs or pumps, and a heating power source. In the distal section, a pair of inflatable balloons including a distal balloon 5 and a proximal balloon 6 are disposed on the catheter body 2 and bound and define a heating segment 7. The catheter body in the heating segment has an outer diameter of about 2 to 5 mm, depending on the initial size and desired post-treatment size of the vein in which the catheter is to be used. The catheter body is preferably a relatively flexible catheter body, to permit percutaneous insertion and navigation through the veins of the patient, although it may be provided as a stiff and inflexible catheter body for treatment of valves readily accessible from nearby percutaneous access sited. The balloons are supplied with fluid through lumens 8 and 9 (they may also be supplied by a single shared lumen). Within the heating segment, a pair of heating elements, including the distal heating element 10 and a proximal heating element 11 are mounted on the outside of the catheter body 2, and electrical wires 12 and 13 running through the catheter body connect the heating element to a source of power through the proximal end of the catheter. The heating elements are comprised of a resistive heating elements, electrodes, RF electrodes, ultrasonic heat sources, LED's and other light or laser sources, or other suitable heating mechanisms. Where the heating elements are resistive heating elements, the electrical wires will comprise a ground wire and a hot wire, and while a minor amount of current may pass through the body to ground, the bulk heating of the venous tissue will be caused by conductive heating from the heating elements which are in turn heated due to resistance of the elements and the passage of current through the elements. Appropriate materials for the resistive heating elements include nichrome and nickel-titanium alloys such as nitinol. One or more suction ports 14 connected to a suction lumen 15 (shown in phantom) within the catheter body communicates with the exterior of the catheter body in the vicinity of the heating segment. The suction port is located relative to the heating elements such that suction applied to the vessel through the suction port will draw the tissue of the vessel near the valve toward the heating elements. When placed within the body as illustrated, the catheter distal segment 3 is inserted into a section of the patient's vein 16 which has a venous valve 17 which is incompetent and requires treatment. The balloons reside on the distal side 18 of the venous valve and the proximal side 19, and when inflated create a segment of isolated vein which includes the venous valve. The distal heating element 10 is located on the distal side 18 of the valve, while the proximal heating element 11 is located on the proximal side 19 of the valve. The proximal and distal sides of the valve are defined here in relation to blood flow within the vein, with the distal side being downstream from the proximal side, or, in other words, in relation to the origin of blood flow. In the illustration, access is gained through a superficial vein upstream of the valve. The proximal and distal components of the catheter would of course be placed in the distal and proximal sides, respectively, if access is gained from a point downstream of the valve and the approach into the vein is retrograde, or upstream, in the blood flow. Proper placement of the catheter relative to the venous valve can be confirmed with ultrasound imaging, endoscopic viewing, palpation by the operating surgeon, or any other means.

Referring now to the proximal section 4 of the catheter body, this section includes a hub 21. The hub includes a luer fitting 22 which is connected to the suction lumen 15. The luer fitting is adapted for air-tight connection to a source of vacuum 23. Shutoff valves 24 and throttle valves 25 may be placed in line between the vacuum and the luer fitting, so that suction can be controlled or terminated as desired by the operator. The hub also includes luer fittings 26 and 27 connected in fluid communication with the inflation lumens 8 and 9, respectively. These luer fittings are adapted for connection to the inflation fluid source 28 which provides pressurized fluid to the system. A shutoff valve 29 and throttle valve 30 may be placed in line between the inflation fluid source and the luer fittings, so that inflation pressure can be controlled or terminated as desired by the operator. The hub also includes the proximal end of the heating element electrical wires 12 and 13, and these are conveniently terminated in an electrical connector 31 which may be integral or separate from the hub. The electrical connector provides for connection of the electrical wires to the direct current power supply 32. The direct current power supply, should be capable of delivering direct current power in the range of 1 to 72 watts, with voltage in the range of 1-12 volts and amperage in the range of 1-6 amps.

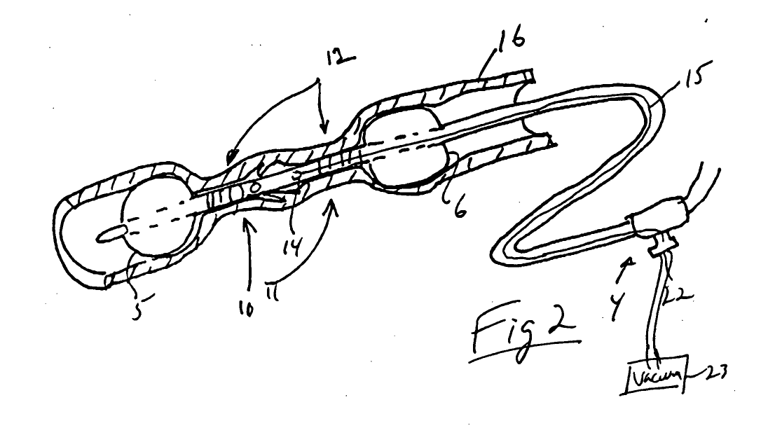

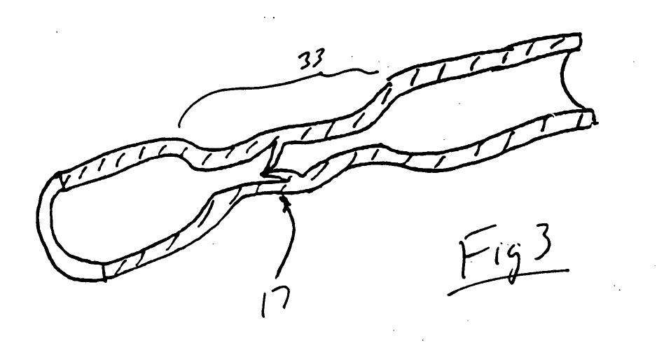

While FIG. 1 illustrates the device components and the initial placement of the catheter within a segment of diseased vein, FIG. 2 illustrates the catheter in operation, drawing the vein segment to be treated toward the catheter body. With the balloon inflation reservoir connected to the inflation luer fittings, the distal and proximal balloons have been inflated to create a cylindrical lumenal space between the balloons having the vein as the wall of the cylinder. Optionally, saline or other solution (such as an anti-coagulant) has been flushed through the vacuum lumen 15 and suction port 14 to flush out the blood within the cylinder or dilute or treat the blood within the cylinder prior to application of suction. The vacuum source 23 has been connected to the vacuum line luer fitting 22, and the valves have been opened to create a vacuum in the cylindrical space between the balloons. Under the vacuum, the vein between the balloons has collapsed upon the catheter body and heating segment 7. In the next step of the procedure, electrical current is applied to the collapsed vein segment through the heating elements, supplied with direct current from the DC source. Preferably, the direct current is applied at power levels adequate to shrink the vein walls on either side of the venous valve, but not high enough to thermally damage the valve itself. Should thermal damage to the valve itself be medically indicated, the power levels may be increased and the heating elements may be located on the catheter body to correspond with the location of the venous valve. After treatment, the catheter may be withdrawn, leaving the vein in the condition illustrated in FIG. 3, where the valve 17 now spans a segment 33 of vein which is constricted vis-à-vis its original state.

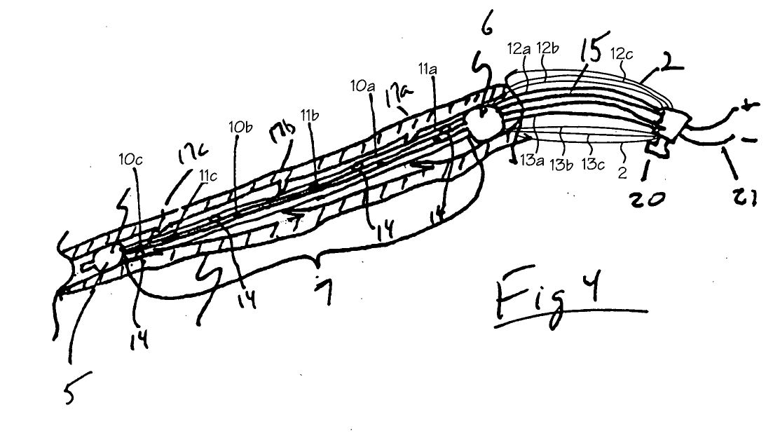

FIG. 4 illustrates a venous repair catheter positioned with a vein, where the catheter is adapted to treat a vein segment including several valves in a single application of heating power. In the distal section, a pair of inflatable balloons including a distal balloon 5 and a proximal balloon 6 are disposed on the catheter body 2 and bound and define a heating segment. The balloons are supplied with fluid through lumens 8 and 9. Within the heating segment 7, a first pair of heating elements, including the distal heating element 10a and a proximal heating element 11a are mounted on the outside of the catheter body 2, and electrical wires 12a and 13a running through the catheter body connect the first heating elements to a source of power through the proximal end of the catheter. These heating elements are located relative to the proximal balloon so that they will be located with on either side of a venous valve 17a upon positioning of the balloon near the venous valve. A second pair of heating elements, including the distal heating element 10b and a proximal heating element 11b are mounted on the outside of the catheter body 2, and electrical wires 12b and 13b running through the catheter body connect the second pair of heating elements to a source of power through the proximal end of the catheter. This second pair of heating elements are located relative to the proximal balloon so that they will be located with on either side of a second venous valve 17b upon positioning of the balloon near the first venous valve. A third pair of heating elements, including the distal heating element 10c and a proximal heating element 11c are mounted on the outside of the catheter body 2, and electrical wires 12c and 13c running through the catheter body connect the third pair of heating elements to a source of power through the proximal end of the catheter. This third pair of heating elements are located relative to the proximal balloon so that they will be located with on either side of a third venous valve 17c upon positioning of the balloon near the first venous valve. As in FIG. 1, one or more suction ports 14 are connected to a suction lumen 15 (shown in phantom) within the catheter body, and provides suction to the exterior of the catheter body in the vicinity of the heating segment. Using this catheter, several incompetent valves and surrounding areas may be treated at the same time.

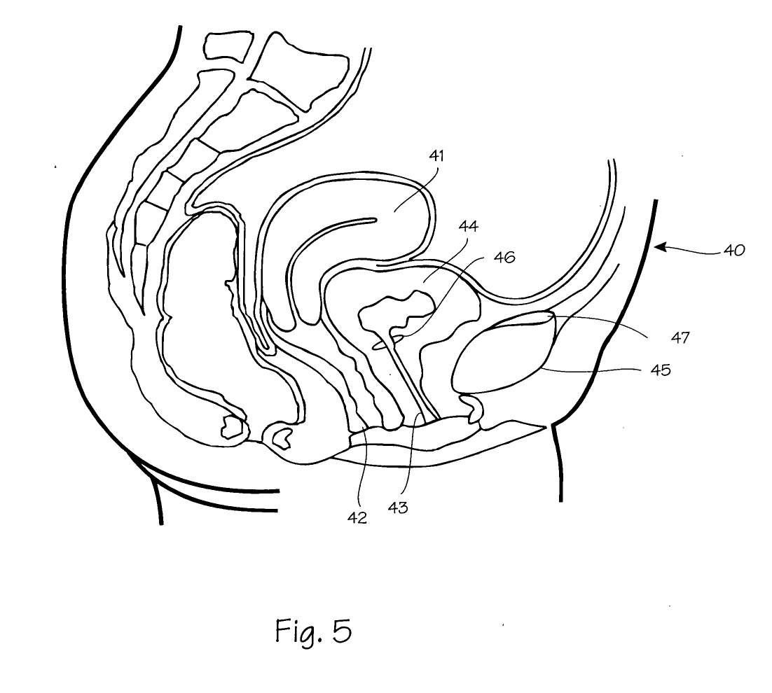

FIG. 5 illustrates the anatomy involved in stress urinary incontinence in a female patient. The typical anatomy of a female patient 40 is illustrated in this sagittal cross section of the pelvic area, and includes anatomical structures such as the uterus 41, the vagina 42 the urethra 43, the bladder 44, and the pubis symphasis 45. The bladder neck sphincter 46 is located at the junction between the bladder and the urethra, at the proximal end of the urethra. In a bladder neck suspension procedure, sutures are used to tie the tissue posterior to the proximal urethra to anterior structures such as the Coopers ligament 47 located above the pubis symphasis 45.

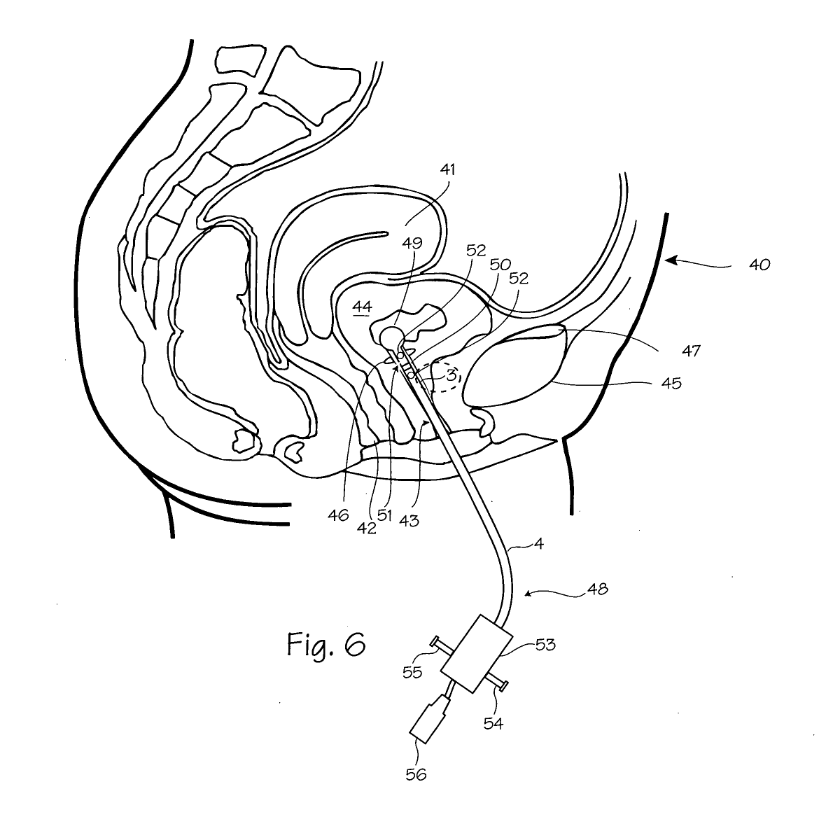

FIG. 6 illustrates the bladder neck sphincter repair catheter 48 adapted for use in treatment or stress urinary incontinence in a female patient. The repair catheter includes the tubular body 2 characterized by a distal section 3, a proximal section 4. The distal section in adapted for insertion into the urethra, and preferably has a distal tip adapted for insertion into the bladder. The proximal end, which remains outside the body, is adapted for connection various external systems, such as to source of vacuum, inflation reservoirs or pumps, and a heating power source, as described in reference to the venous valve repair catheter. In this embodiment, a single balloon 49 is mounted at the distal tip of the catheter, and a heating element 50 is mounted a short distance proximal to the balloon, spaced from the balloon a distance 51 chosen to facilitate and ensure placement of the heating element distal (relative to the bladder and urethra) to the bladder neck sphincter, but still fairly close to the bladder neck sphincter and in the proximal portion of the urethra. Also on the distal end of the catheter, proximal to the balloon and located in the vicinity of the heating element, one or more suction ports 52 in communication with the exterior of the catheter may be provided. The catheter body in the heating segment has an outer diameter of about 2 to 5 mm, depending on the initial size and desired post-treatment size of the urethra in which the catheter is to be used. The catheter body is preferably a relatively stiff catheter body, to permit insertion through the urethra. The proximal end of the catheter is provided with a hub 53 fitted inflation luer 54 and suction luer connection 55, should suction be required to draw the urethra to the catheter body, and an electrical connector 56 for providing power to the heating element. Within the catheter body 2, the requisite inflation lumen and suction lumen connect the balloon and suction ports to their respective luer connections, and electrical leads connect the heating element to the electrical connector on the proximal hub.

In use, the operator inserts the catheter of FIG. 6 into the urethra, until the balloon enters the bladder. The operator then inflates the balloon so that it impedes pullout of the catheter from the urethra, and then seats the proximal surface of the balloon on the urethral opening into the bladder. This should locate the heating element just distal to the bladder neck sphincter, and the operator should confirm this with ultrasound imaging, palpation, fluoroscopy, MRI or other imaging means. With the heating element properly placed, the operator applies direct current energy through the heating element to the urethral wall distal to the bladder neck sphincter. This heating will then result in shrinkage of the urethral section distal to the bladder neck sphincter. The effect of the bladder neck suspension will thus be achieved without sutures or surgery. If the diameter of the urethra exceeds the diameter of the catheter body in the heating segment, the operator may apply suction to the urethra through the suction ports on the catheter and thereby draw down the proximal urethra onto the catheter body. After drawing down the urethra to the diameter of the catheter body, the direct current energy is applied to the heating elements. The power supply, when used to treat the proximal urethra, should be capable of delivering direct current power in the range of 1 to 72 watts, with voltage in the range of 1-12 volts and amperage in the range of 1-6 amps.

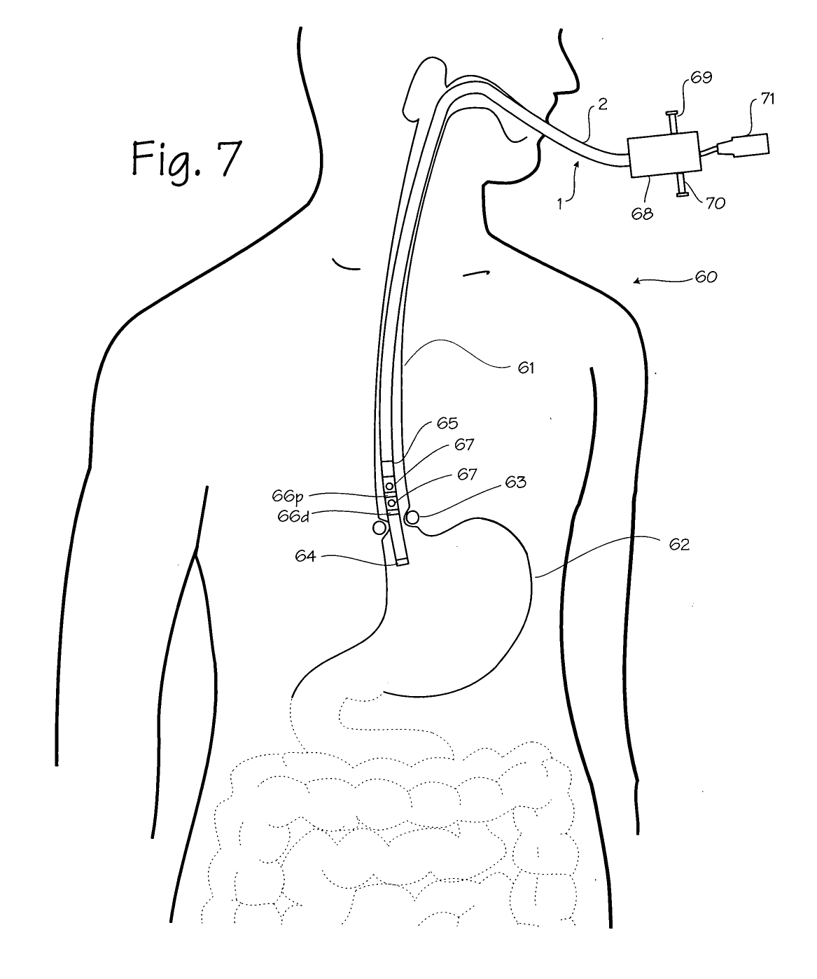

FIG. 7 illustrates the anatomy involved in gastroesophageal reflux disease, and its relationship to the parts of the repair catheter. The patient 60 is shown with the repair catheter 1 extending from outside the body, entering the mouth and extending down the esophagus 61 until the distal tip extends into the stomach 62. When properly positioned, the heating segment will be positioned above (proximal to) the lower esophageal valve 63. The distal balloon 64 is located at the tip of the catheter so that it may be inflated, as shown, within the stomach and below the lower esophageal sphincter. The proximal balloon 65 is located proximal to the heating segment, so that it can be inflated as shown within the esophagus to seal off a cylindrical space between the proximal balloon and the distal balloon. One or more heating elements 66 are mounted on the heating segment. Also on the distal end of the catheter, proximal to the distal balloon and located in the vicinity of the heating element, one or more suction ports 67 in communication with the exterior of the catheter may be provided. The catheter body in the heating segment has an outer diameter of about 15 to 25 mm, depending on the initial size and desired post-treatment size of the esophagus in which the catheter is to be used. The catheter body is preferably a sufficiently flexible to permit negotiation of through the mouth and into the esophagus. The proximal end of the lower esophageal sphincter repair catheter is provided with a hub 68 fitted inflation luer 69 and suction luer connection 70, should suction be required to draw the urethra to the catheter body, and an electrical connector 71 for providing power to the heating element. Within the catheter body 2, the requisite inflation lumens and suction lumen connect the balloon and suction ports to their respective luer connections, and electrical leads connect the heating element to the electrical connector on the proximal hub.

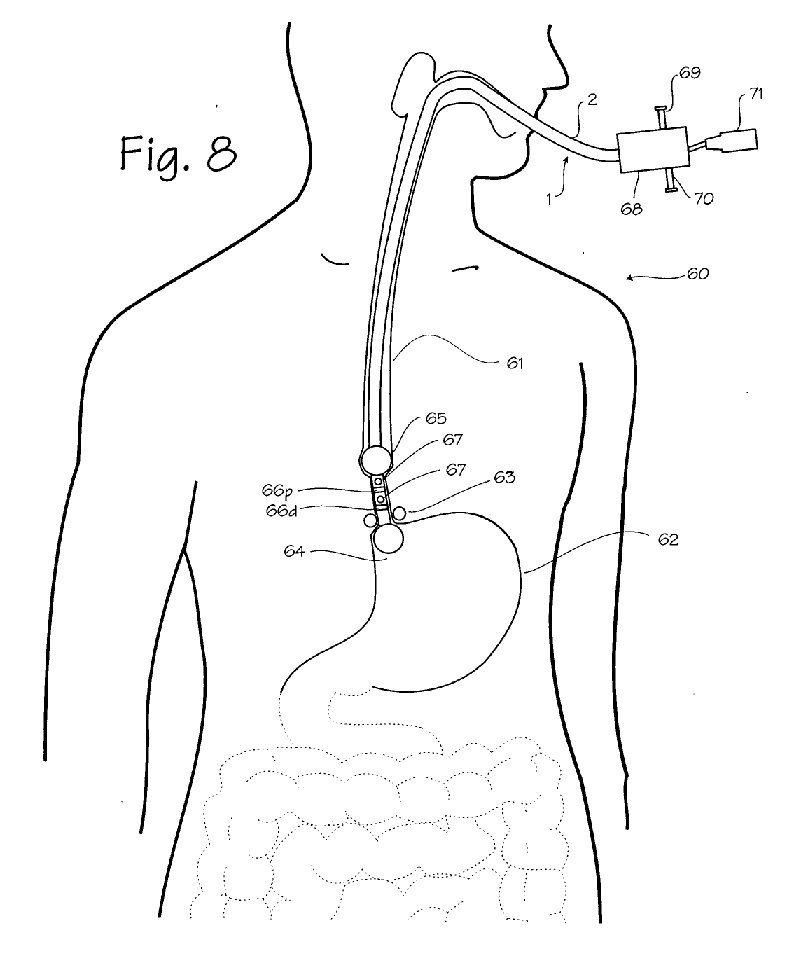

FIG. 8 illustrates the repair catheter in use in the treatment or gastroesophageal reflux disease in a patient. The distal balloon 64 and the proximal balloon 65 have been inflated to seal off a cylindrical segment of the lower esophagus. Vacuum has been applied through the suction ports 67, drawing the esophagus surrounding the heating segment into close proximity of the heating element(s) 66. With the esophageal tissue immediately above the lower esophageal sphincter drawn down to the heating segment, the operator can energize the heating element, with direct current passed through the connector in the hub, to cause ablation of the esophagus just above the lower esophageal sphincter. The direct current power supply, when used to treat the lower esophagus, should be capable of delivering direct current power in the range of 1 to 72 watts, with voltage in the range of 1-12 volts and amperage in the range of 1-6 amps.

Determination of the endpoint of the treatment may be accomplished in several ways. Temperature sensors may be placed on the outer surface of the heating segment, and application of the heating power may be limited to maintain a temperature, based on feedback from the temperature sensors, at the surface of the probe in the range of about 45-50° C. Total power applied to the tissue supporting the valve can then be controlled by limiting the duration or time period in which power is applied. Additionally, suction and heating may be interrupted, and the vessel observed through ultrasound or other imaging technique. When the operator observes that the vessel has shrunk to the desired size, treatment may be halted and deemed complete.

Direct current, applied to the resistive heating elements, has been discussed as the preferred power source for applying thermal energy to the structures surrounding and supporting anatomical valves. Other power source may be used, such as alternating current and radiofrequency current (RF). RF power may be applied in bipolar mode or monopolar modes. For bipolar application, RF energy will flow from one electrode on the catheter to another, such as from electrode 66d and 66p shown in FIGS. 7 and 8. For monopolar RF application, a ground electrical on the surface of the patient's body must be provided, and RF energy will flow from each electrode 66d and 66p to the surface ground electrode. Various other sources of ablative or injurious power may be used, including lower frequency AC electrical power, ultrasound energy, radiation, cryosurgical devices and chemical ablating agents. The energy is applied to damage or injure tissue in the body of the vessel that supports the valve which controls flow of fluids through the vessel. This tissue in the body of the vessel may be distal to the valve, proximal to the valve, or both. Preferably, the valve itself is not injured unless injury is indicated for additional treatment of the incompetence.

Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

I claim:

1. A method of treating a vessel of the human body, wherein the vessel includes an anatomical valve or sphincter which controls the flow of fluids through the vessel, said method comprising:
providing a catheter having a distal end adapted for insertion into the vessel, a first balloon disposed on the distal end of the catheter, a heating element disposed on the distal end of the catheter a short distance proximal to the balloon and a suction port located near the heating element;
inserting the distal end of the catheter into the vessel so that the heating element is located near the valve or sphincter;
inflating the first balloon;
applying suction to the vessel through the suction port to draw down the vessel wall until the vessel is in contact with the heating element;
heating the heating element to cause thermal injury to the vessel; and
withdrawing the catheter from the vessel.

2. The method of claim 1 further comprising the steps of:
providing a second balloon on the distal end of the catheter, said second balloon located proximal to the first balloon, the heating element and the suction port;
after inserting the distal end of the catheter into the vessel, inflating the second balloon to isolate the section of the vessel including the valve or sphincter between the first and second balloons.

3. The method of claim 1 further comprising:
flushing a fluid through the suction port into the vessel prior to applying suction to the vessel through the suction port.

4. The method of claim 2 further comprising:
flushing a fluid through the suction port into the vessel prior to applying suction to the vessel through the suction port.

5. The method of claim 1 wherein the step of heating the heating element further comprises limiting the heat emanated by the heating element to avoid injuring the valve or sphincter.

6. The method of claim 1 wherein the step of providing a catheter further comprises providing a catheter wherein the short distance between the heating element and the balloon is set to facilitate placement of the heating element a short distance distal to a bladder neck sphincter, relative to a bladder and a urethra in a female patient.

7. A method of treating a vessel of the human body, wherein the vessel includes an anatomical valve or sphincter which controls the flow of fluids through the vessel and is supported by the tissue of the vessel near the valve or sphincter, said method comprising:
providing a device for treating an incompetent anatomical valve or sphincter within the body of a patient, said device comprising:
a catheter body having a distal end and a proximal end, said distal end being adapted for insertion into the body;
a first balloon located at the distal end of the catheter, said first balloon being inflatable to a diameter greater than the catheter body distal end, and a first inflation lumen communicating from the proximal end of the catheter body to the distal end of the catheter body, wherein the first inflation lumen is in fluid communication with the first balloon;
a first heating element mounted on the distal end of the catheter, proximal to the first balloon, said first heating element capable of delivering sufficient energy to the tissue of the vessel near the valve or sphincter to shrink the tissue near the valve or sphincter;
a second balloon located at the distal end of the catheter, said second balloon being inflatable to a diameter greater than the catheter body distal end, said second balloon proximal to the first balloon and proximal to the first heating element, and a second inflation lumen communicating from the proximal end of the catheter body to the distal end of the catheter body, wherein the second inflation lumen is in fluid communication with the second balloon;
a second heating element mounted on the distal end of the catheter, distal to the second balloon and proximal to the first heating element, said second heating element capable of delivering sufficient energy to the tissue of the vessel near the valve or sphincter to shrink the tissue near the valve or sphincter;
a suction lumen communicating from the proximal end of the catheter body to the distal end of the catheter body, and at least one suction port located on the distal end of the catheter communicating from the suction lumen to the exterior of the catheter body, said at least one suction port being located proximal to the first heating element and distal to the second heating element;
inserting the distal end of the catheter into the vessel so that the valve or sphincter is disposed between the first heating element and the second heating element;
inflating the first balloon and the second balloon;
applying suction to the vessel through the at least one suction port to draw down the vessel wall until the vessel is in contact with the first heating element and the second heating element;
heating the first heating element and the second heating element to cause thermal injury to the vessel; and
withdrawing the catheter from the vessel.

8. The method of claim 7 wherein the step of providing a device further comprises providing the first heating element and second heating element in the form of resistive heating elements.

9. The method of claim 7 comprising the further step of flushing a fluid through the at least one suction port into the vessel prior to applying suction to the vessel through the at least one suction port.

10. The method of claim 7 wherein the step of heating the first heating element and the second heating element further comprises limiting the heat emanated by the first heating element and by the second heating element to avoid injuring the valve or sphincter.

11. A method of treating a vessel of the human body, wherein the vessel includes a plurality of anatomical valves or sphincters which control the flow of fluids through the vessel and are supported by the tissue of the vessel near the plurality of valves or sphincters, said method comprising:
providing a device for treating a plurality of incompetent anatomical valves or sphincters within the body of a patient, said device comprising:
a catheter body having a distal end and a proximal end, said distal end being adapted for insertion into the body;
a first balloon located at the distal end of the catheter, said first balloon being inflatable to a diameter greater than the catheter body distal end, and a first inflation lumen communicating from the proximal end of the catheter body to the distal end of the catheter body, wherein the first inflation lumen is in fluid communication with the first balloon;
a second balloon located at the distal end of the catheter, said second balloon being inflatable to a diameter greater than the catheter body distal end, said second balloon proximal to the first balloon, and a second inflation lumen communicating from the proximal end of the catheter body to the distal end of the catheter body, wherein the second inflation lumen is in fluid communication with the second balloon;
a plurality of heating elements mounted on the distal end of the catheter body, wherein each of the plurality of heating elements are disposed in series along the length of the catheter body, wherein two succeeding heating elements comprise a pair of heating elements, and wherein each pair of heating elements is further disposed on the catheter body such that a section of catheter body separates each pair of heating elements, wherein each of the plurality of heating elements is capable of delivering sufficient energy to tissue of the vessel near the valves or sphincters to shrink the tissue near the valves or sphincters;
a suction lumen communicating from the proximal end of the catheter body to the distal end of the catheter body; and
a plurality of suction ports located on the distal end of the catheter communicating from the suction lumen to the exterior of the catheter body, wherein at least one of the plurality of suction ports is disposed between each pair of heating elements;
inserting the distal end of the catheter into the vessel so that each of the plurality of valves or sphincters is disposed between a corresponding pair of heating elements;
inflating the first balloon and the second balloon;
applying suction to the vessel through the plurality of suction ports to draw down the vessel wall until the tissue of the vessel near the plurality of valves or sphincters is in contact with the plurality of heating elements;
heating the plurality of heating elements to cause thermal injury to the vessel; and
withdrawing the catheter from the vessel.

12. The method of claim 11 wherein the step of providing a device further comprises providing the plurality of heating elements in the form of resistive heating elements.

13. The method of claim 11 comprising the further step of flushing a fluid through the plurality of suction ports into the vessel prior to applying suction to the vessel through the plurality of suction ports.

14. The method of claim 11 wherein the step of heating the plurality of heating elements further comprises limiting the heat emanated by the plurality of heating elements to avoid injuring the plurality of valves or sphincters.
